(19) United States
(12) Patent Application Publication
(10) Pub. No.: US 2025/0135153 A1
(43) Pub. Date: May 1, 2025

(54) PERIPHERALLY INSERTED CENTRAL CATHETERS (PICC) AND METHODS OF USE THEREOF TECHNICAL FIELD

(71) Applicant: Alon SHIRI, Bnei-Brak (IL)

(72) Inventor: Yaacov BARNEA, Singapore (SG)

(21) Appl. No.: 18/683,625

(22) PCT Filed: Aug. 21, 2022
(86) PCT No.: PCT/IL2022/050912

§ 371 Date: Feb. 14, 2024

Related U.S. Application Data
(60) Provisional application No. 63/236,538, filed on Aug. 24, 2021.

(51) Int. Cl. A61M 25/00; A61M 39/00; A61M 39/22

(52) U.S. Cl. CPC A61M 25/0026; A61M 39/22; A61M 2039/0018

(57) ABSTRACT

There is provided herein a peripherally insertable central catheter (PICC) comprising a distal portion, configured to be inserted into a blood vessel, the distal portion comprises a distal end face opening; a proximal portion comprising a proximal opening; and at least one flushing lumen extended therebetween; a valve positioned within the at least one flushing lumen, the valve comprises two configurations, an open configuration, allowing fluid flow through the distal end face opening, and a closed configuration, blocking fluid flow through the distal end face opening, wherein at a closed configuration of the valve, flushing fluid that is inserted to the catheter from the proximal opening thereof, circulates back through the proximal portion and out of the subject's blood vessel, thereby exiting of flushing fluid through the distal end face opening is prevented.

The present disclosure relates generally to Peripherally Inserted Central Catheters (PICC) and methods of use thereof.

### BACKGROUND

Catheter is a long-established medical device based on a hollow lumen generally introduced into a body organ or a blood vessel for draining fluids, delivery of medical substances, nutrition, monitoring and other medical-related procedures. Peripherally Inserted Central Catheters (PICC) is a family of catheters typically implemented for a long-term medical intravenous (IV) therapy, such as cancer treatment. Generally, the insertion of the PICC is performed via a peripheral arm vein using nitinol guidewire and X-ray imaging. Inserted PICC devices may be remained inside a patient's body for a prolonged period in some cases of more than 6months. Such a long duration of usage adds to the risk of PICC causing infections, theses may include external bacteria dwelling into the body, for instance, during the administration of the device, and/or due to gap presence between the PICC and the body, internal bacteria or contaminations presence. Catheter-related bloodstream infections, bacteria growth, as well as blood clotting, occlusion, precipitates deposition, air embolism, disposition, leakage, kinks presence and other catheter malfunctions are highly associated with deterioration of the patient's condition and may even lead to mortality.

Flushing of the PICC device with a cleaning solution, such as saline, is a commonly applied method performed routinely, also following medical treatments, for removing undesired substances and bacteria from the catheter lumen and maintaining the catheter patency. Adequately performed flushing procedure is of great importance for preventing the accumulation of the undesired substances inside the catheter lumen.

Nevertheless, there is a need in the art for an improved PICC design and flushing method for facilitating disinfecting and removing the undesired substances from the catheter lumen.

### SUMMARY

Aspects of the disclosure, according to some embodiments thereof, relate to a catheter, and specifically but not exclusively to a peripherally insertable central catheter (PICC) for administration fluids, such as medical substances and nutrition, into the subject. Following the delivery of the medical substances into a blood vessel and/or body organ, the PICC is regularly cleaned by introducing a flushing fluid, such as saline, into the catheter. Although the flushing fluid minimizes accumulation of bacteria, clotting factors, and the like, inside the catheter, the flushing fluid is typically drained into the subject's body, administrating undesired substances into the subject. However, the special design of the current disclosure includes a PICC with at least one flushing lumen, and a valve positioned within the at least one flushing lumen. The valve comprises two configurations, an open configuration, allowing fluid flow through the distal end face opening, and a closed configuration, blocking the fluid flow through the distal end face opening. At the closed configuration of the valve, flushing fluid that is inserted into the catheter from the proximal opening is circulated back though the proximal portion and out of the subject's blood vessel, preventing the exiting of the flushing fluid though the distal end face opening. The disclosed device allows draining of the circulated flushing fluids outside of the blood vessel and/or body organ. Thus, the current disclosure enables further analysis of the flushing fluid for identification of contaminations, bacteria, and other undesired substances. Further, the valve enables controllable and rapid transitions between the open and the closed configurations, providing a closed circulating system for a bidirectional and circulated flow of the flushing fluid, therefore significantly decreasing the risk of infections and accumulation of deposits inside the catheter. The closure of the PICC, when not operational, may also assist in prevention of dwelling bacteria caused by backpressure and movement of the PICC inside the body.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

According to some embodiments, the at least one flushing lumen may be divided into a plurality of individual lumens. According to some embodiments, the at least one flushing lumen may be divided into two individual lumens, a first lumen configured to deliver the flushing fluid, and a second lumen configured to collect the flushing fluid and direct it to the proximal portion and out of the subject's blood vessel. Thus, preventing the delivery of the flushing lumen including contaminations, precipitates, clotting factors, bacteria, and the like into the subject's body. According to some embodiments, the at least one flushing lumen may be divided into three individual lumens, wherein a third lumen is configured to deliver a medical substance to the subject.

According to some embodiments, the valve may comprise an inflatable element configured to inflate into dimension sufficient for sealing the distal end face opening. According to some embodiments, the inflatable element may be a balloon. According to some embodiments, the catheter may include an inflation conduit in fluid flow communication with the inflatable element. According to some embodiments, inflation and deflation of the inflatable element may be performed via the inflation conduit.

According to some embodiments, the valve may include a sealing flap and/or a shutter configured to controllably seal the distal end face opening.

According to some embodiments, the catheter may include one or more sensors for monitoring and/or regulating pressure and/or capacity of fluids (e.g., flushing fluid, medical substances) entering and/or exiting the catheter.

Also disclosed herein is a method for flushing of a PICC. The method includes providing a PICC, as previously disclosed, and inserting the distal position of the PICC into a blood vessel and/or body organ of a subject. The method also includes shifting the valve to a closed configuration for blocking fluid flow through the distal end face opening, and injecting flushing fluid to the catheter from the proximal opening thereof, thereby the flushing fluid circulates back through the proximal portion and out of the catheter such that exiting of flushing fluid through the distal end face opening and into the subject's blood vessel is prevented.

According to some embodiments, the method further includes analyzing the flushing fluid for chemical and/or biological substances indicative of complications and/or infections. According to some embodiments, analyzing of the flushing fluid may include identification of blood clots and/or bacteria.

According to some embodiments, the method may include analyzing one or more parameters associated with a pressure of the flushing fluid exiting and/or entering the catheter for assessing the performance quality of the catheter and/or the flushing fluid.

According to some embodiments, the method may include monitoring and/or regulating pressure of the flushing fluid using one or more sensors.

According to some embodiments, the method may include monitoring and/or regulating flow capacity of the medical substance and/or flushing fluid entering the blood vessel using one or more sensors.

According to some embodiments, the method may include regulating, utilizing a controller, pressure and/or flow capacity of the flushing fluid and/or the medical substance.

According to some embodiments there is provided a peripherally insertable central catheter (PICC) including a distal portion, configured to be inserted into a blood vessel, the distal portion includes a distal end face opening, a proximal portion including a proximal opening, at least one flushing lumen extended therebetween, and a valve positioned within the at least one flushing lumen, the valve includes two configurations, an open configuration, allowing fluid flow through the distal end face opening, and a closed configuration, blocking fluid flow through the distal end face opening, wherein at a closed configuration of the valve, flushing fluid that is inserted to the catheter from the proximal opening thereof, circulates back through the proximal portion and out of the subject's blood vessel, thereby exiting of flushing fluid through the distal end face opening is prevented.

According to some embodiments there is provided a method for flushing a peripherally insertable central catheter (PICC), the method including: providing a PICC including a distal portion including a distal end face opening, a proximal portion including a proximal opening, at least one flushing lumen extended therebetween, and a valve positioned within the at least one flushing lumen, inserting the distal portion of the PICC into a blood vessel of a subject, shifting the valve to a closed configuration for blocking fluid flow through the distal end face opening, and injecting flushing fluid to the catheter from the proximal opening thereof, thereby the flushing fluid circulates back through the proximal portion and out of the catheter such that exiting of flushing fluid through the distal end face opening and into the subject's blood vessel is prevented.

According to some embodiments, the at least one flushing lumen is further configured to deliver a medical substance to the subject at an open configuration of the valve.

According to some embodiments, the at least one flushing lumen is divided into two individual lumens, a first lumen configured to deliver the flushing fluid and a second lumen configured to collect the flushing fluid and direct it to the proximal portion and out of the subject's blood vessel.

According to some embodiments, the valve includes an inflatable element configured to inflate into dimensions sufficient for sealing the distal end face opening.

According to some embodiments, the catheter further includes an inflation conduit in fluid flow communication with the inflatable element.

According to some embodiments, the valve includes a sealing flap and/or a shutter configured to controllably seal the distal end face opening.

According to some embodiments, the valve includes a duckbill valve configured to controllably seal the distal end face opening.

According to some embodiments, the valve may include a skirt valve configured to controllably seal the distal end face opening.

According to some embodiments, the valve may include a guidewire valve configured to controllably seal the distal end face opening. According to some embodiments, the guidewire valve is a valve operated by a guidewire such that upon pushing the guidewire distally the guidewire valve is shifted to a closed configuration, thereby sealing a distal end face a PICC, and upon pulling the guidewire proximately the guidewire valve is shifted to an open configuration, thereby allowing fluid flow through a distal opening of the distal end face of the PICC.

According to some embodiments, the valve may include a piston configured to controllably seal the distal end face opening. According to some embodiments, the piston may be operated by a wire such that upon pushing the wire distally the piston is shifted into a closed configuration, thereby sealing a distal end face a PICC, and upon pulling the piston proximately the piston is shifted to an open configuration, thereby allowing fluid flow through a distal opening of the distal end face of the PICC.

According to some embodiments, the catheter further includes an additional individual lumen for delivering a medical substance to the subject at an open configuration of the valve.

According to some embodiments, the flushing fluid circulated back through the proximal portion exits the catheter through the proximal opening.

According to some embodiments, the flushing fluid circulated back through the proximal portion exits the catheter through a proximal drain port.

According to some embodiments, the catheter further includes a pressure adjustment mechanism configured to regulate a flow rate of the flushing fluid to facilitate circulation of the flushing fluid through the at least one flushing lumen, back through the proximal portion and out of the catheter.

According to some embodiments, the catheter includes a flushing lumen, a lumen for operating the valve, a lumen for delivering medical substance, and a lumen including a duckbill valve for facilitating the separation of the medical substances,

According to some embodiments, the catheter further includes one or more sensors for monitoring and/or regulating pressure of the flushing fluid to facilitate pressure regulation.

According to some embodiments, the catheter further includes one or more sensors for monitoring and/or regulating flow capacity of the flushing fluid entering and/or exiting the catheter.

According to some embodiments, the catheter further includes one or more sensors for monitoring and/or regulating flow capacity of the medical substance entering the blood vessel.

According to some embodiments, the method further includes analyzing the flushing fluid for chemical and/or biological substances indicative of complications and/or infections.

According to some embodiments, the analyzing the flushing fluid includes identification of blood clots and/or bacteria.

According to some embodiments, the method further includes analyzing one or more parameters associated with a pressure of the flushing fluid exiting and/or entering the catheter for assessing the performance quality of the catheter and/or the flushing fluid.

According to some embodiments, the method further includes monitoring and/or regulating pressure of the flushing fluid using one or more sensors.

According to some embodiments, the method further includes monitoring and/or regulating flow capacity of the flushing fluid entering and/or exiting the catheter using one or more sensors.

According to some embodiments, the method further includes monitoring and/or regulating flow capacity of the medical substance entering the blood vessel using one or more sensors.

According to some embodiments, the method further includes regulating, utilizing a controller, pressure and/or flow capacity of the flushing fluid and/or the medical substance.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not drawn to scale. Moreover, two different objects in the same figure may be drawn to different scales. In particular, the scale of some objects may be greatly exaggerated as compared to other objects in the same figure.

In block diagrams and flowcharts, optional elements/components and optional stages may be included within dashed boxes.

In the figures:

FIG. 1 shows a schematic illustration of a prior art PICC introduced into a subject's blood vessel;

FIG. 2 schematically shows a prior art PICC;

FIG. 3 schematically shows a side view of a distal portion of a PICC with a valve, according to some embodiments;

FIG. 4A schematically shows a perspective view of a distal portion of a PICC, according to some embodiments;

FIG. 4B schematically shows a cross-section view of the distal portion and the distal end face of the PICC of FIG. 4A, according to some embodiments;

FIG. 5A schematically shows a perspective view of a distal portion of a PICC in an open valve configuration, according to some embodiments;

FIG. 5B schematically shows a perspective view of the distal portion of the PICC of FIG. 5A, in closed valve configuration, according to some embodiments;

FIG. 6A schematically shows a cross-section side view of a distal portion of a PICC in an open valve configuration, according to some embodiments;

FIG. 6B schematically shows a cross-section side view of a distal portion of a PICC in a closed valve configuration, according to some embodiments;

FIG. 7A schematically shows a cross-section side view of a distal portion of a PICC in an open valve configuration, according to some embodiments;

FIG. 7B schematically shows a cross-section side view of a distal portion of a PICC in a closed valve configuration, according to some embodiments;

FIG. 8A schematically shows a cross-section side view of a distal portion of a PICC in an open valve configuration, according to some embodiments;

FIG. 8B schematically shows a cross-section side view of a distal portion of a PICC in a closed valve configuration, according to some embodiments;

FIG. 9A schematically shows a cross-section side view of a distal portion of a PICC in an open valve configuration, according to some embodiments;

FIG. 9B schematically shows a cross-section side view of a distal portion of a PICC in a closed valve configuration, according to some embodiments; and

FIG. 10 shows a flow chart of a method of flushing of a PICC, according to some embodiments.

### DETAILED DESCRIPTION

The principles, uses and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the following description, various aspects of the invention will be described. For the purpose of explanation, specific details are set forth in order to provide a thorough understanding of the invention. However, it will also be apparent to one skilled in the art that the invention may be practiced without specific details being presented herein. Furthermore, well-known features may be omitted or simplified in order not to obscure the invention.

The present invention, in some embodiments thereof, relates to catheters and, more particularly, to peripherally inserted central catheters (PICC), to a method and apparatus for inserting and draining fluids to blood vessel and/or body organs.

The disclosed PICC, according to some embodiments, is configured to deliver a medical substance to the subject at an open valve configuration. The medical substance may include drugs, such as cytotoxic drugs for cancer treatment, antibiotic medicines, blood, nutrition, and the like.

According to some embodiments, the disclosed PICC may by implemented for medical substance delivery, optionally for a simultaneous delivery of several medical substances, for preventing undesired mixing of the medical substances before entering the blood vessel and/or body organ. Following the delivery of the medical substances, flushing of the PICC may by performed. An aspect of some embodiments of the present invention relates to a catheter including a PICC having a distal portion, a proximal portion, a valve, and at least one flushing lumen extended in between. The valve, positioned within the at least one flushing lumen, has at least two configurations, an open configuration, allowing fluid flow through the distal end face opening, and a closed configuration, blocking fluid flow through the distal end face opening. Thus, in the closed valve configuration, the fluid inserted through the proximal end face opening is circulated back and exits the catheter through the proximal drain port. The exiting fluid, e.g., the circulated fluid, may be further analyzed for identification of bacteria, clotting factors and contaminations, for assessing the catheter function and/or the subject's medical condition.

According to some embodiments, the at least one flushing lumen may be a lumen within which fluid may flow in at least one direction between the distal end face opening and the proximal drain port. According to some embodiments, the at least one flushing lumen may be configured to enable fluid flow in at least two directions between the distal end face opening and the proximal drain port. According to some embodiments, the at least one flushing lumen may include a plurality of individual lumens. According to some embodiments, the at least one flushing lumen may be divided into a plurality of individual lumens. According to some embodiments, the at least one flushing lumen may be divided into a plurality of individual lumens, wherein a first lumen may be configured to deliver the flushing fluid, and a second lumen may be configured to receive the flushing fluid and direct it to the proximal portion and out of the subject's blood vessel and/or body organ. According to some embodiments, the plurality of individual lumens may be in fluid communication.

According to some embodiments, the at least one flushing lumen includes a valve. According to some embodiments, the valve may include any element configured to prevent entering and/or exiting of fluids from the PICC assembly to the blood vessel and/or the vice versa.

According to some embodiments, the valve positioned within the at least one flushing lumen, may be located inside the distal portion inside of the PICC. According to some embodiments, the valve may be positioned at the distal end face of the PICC. According to some embodiments, and as described in greater detail elsewhere herein, the valve may be positioned between a cavity defined by closed configuration of the valve, an inner wall of the PICC, and the distal end of the plurality of individual lumens, allowing circulating of fluids inside the cavity at the distal portion of PICC, and outside the plurality of individual lumens. Following the circulation, the fluids (e.g., flushing fluids) are directed back towards the proximal end and out of the catheter-outside the subject's body.

According to some embodiments, the valve may include an inflatable element, such as a balloon, configured to inflate into dimensions sufficient for sealing the distal end face opening, thus preventing fluid flow into a blood vessel and/or body organ. According to some embodiments, sealing of the PICC may require the inflatable element to be fully or partially inflated (when in closed configuration). According to some embodiments, the inflatable element, when in closed (inflated) configuration, may be positioned fully and/or partially inside, or fully outside the distal end face of the PICC. According to some embodiments, the valve may include a ring located on the outer and/or inner circumference (e.g., external and/or internal ring) of the at least one flushing lumen, wherein the ring is configured to shift between open and closed configurations. For example, the ring may govern the closed configuration by reducing the circumference of the at least one flushing lumen until no fluid can enter and/or exit the distal end face of the PICC assembly; the ring may govern the open configuration by increasing the circumference of the flushing lumen, allowing entering and/or exiting fluids through the distal end face. According to some embodiments, the at least one flushing lumen may comprise a groove configured to retain the ring, providing a smooth outer surface of the flushing lumen. A smooth external and/or internal surface area is beneficial for minimizing the accumulation of undesired substances and deposits on the external and/or internal surface areas of the lumen, for decreasing the risk of infections, bacteria growth, and various contaminations.

According to some embodiments, the distal portion of the PICC assembly may include an angular distal end face. The angular distal end face may include a conus-like shape, wherein the diameter of the at least one flushing lumen is decreasing at the distal end face. The valve may be located at the conus-like shape of the distal end face, wherein the valve is configured to shift from the open and the closed configurations (and vice versa), as required. Valve configuration may be governed mechanically, for example, by applying a mechanical pressure positioning and retaining the valve at the narrowest region of the conus-like configuration, e.g., at the angular distal end face.

According to some embodiments, the valve may include a sealing flap and/or a shutter, configured to controllably seal the distal end face opening. Preferably, the scaling will occur rapidly, minimizing leakage from and/or into the catheter. Optionally, a service conduit may be permanently positioned at the at least one flushing lumen, for example at a service lumen, at the distal portion of the PICC. Optionally or alternatively, the service conduit may be temporarily positioned inside the at least one flushing lumen, and thus may be removed following the shifting of the valve configuration. The service conduit may be elongated, reaching the proximal end face of the PICC assemble, thereby allowing an external control of the valve configuration, for example, for controlling and/or applying mechanical pressure on the valve. According to some embodiments, service conduit positioned inside service lumen may prevent fluid communication of the service lumen with the at least one flushing lumen of the PICC. Put differently, when the service lumen is associated with the service conduit (e.g., for inflation/deflation of an inflatable element by a gas flow) no fluids may enter and/or exit the service lumen.

According to some embodiments, the service conduit may be associated with the valve, wherein sealing of the distal end face is performed by reducing the diameter of the at least one flushing lumen until no gap is present at the distal end face, preventing penetration of fluids.

According to some embodiments, the catheter may include a pressure adjustment mechanism, configured to regulate and/or monitor a flow rate of the flushing fluid to facilitate circulation of the flushing fluid through the at least one flushing lumen. Furthermore, since the volume of the administrated fluids is relatively high with respect to the surface area of the catheter-a long and a thin lumen-pressure adjustment mechanism may be needed for providing the optimal power flow. The pressure adjustment mechanism may include, for example, a pump and/or pumping system for the entering and/or exiting fluids.

According to some embodiments, the catheter may include a valve, e.g., a balloon valve, a flushing lumen, a service lumen, and a lumen with a duckbill valve. The flushing lumen may be utilized for nutrition supplements delivery and/or for flushing purposes, the service lumen may be utilized for the valve configuration operating, and the lumen with the duckbill valve may be applied for administrating e.g., cytotoxic drugs to the subject. The duckbill valve may also include any other type of a controlled valve for regulating the entering and/or exiting fluids, and for facilitating the separation of the medical substances and minimizing residue formation inside the catheter. Moreover, the operating mechanism of the duckbill valve is technically simple, thus it may be beneficial for the subject to use such a catheter, allowing an independent operating that does not require presence at a medical facility.

Optionally, in some embodiments, the catheter may include one or more sensors, allowing pressure regulation and/or monitoring of the flushing fluid, thereby facilitating pressure regulation inside the lumens of the catheter. In some embodiments, one or more sensors may regulate and/or monitor the flow capacity of the flushing fluid and/or the medical substances entering and/or exiting the catheter.

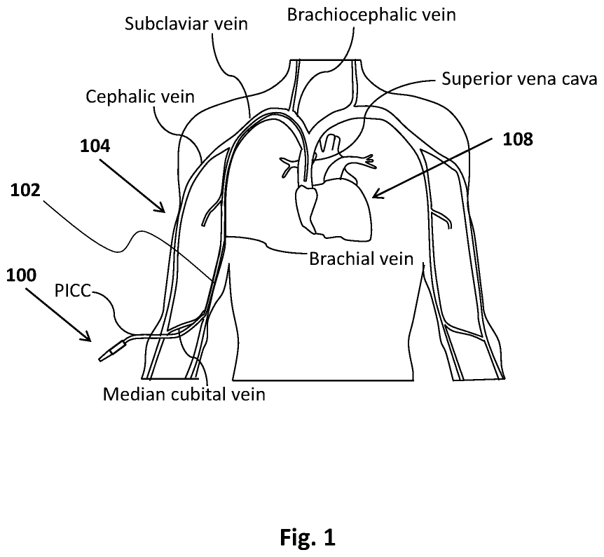

Reference is now made to FIG. 1, which schematically shows a peripherally insertable central catheter (PICC) 100 introduced into a blood vessel 102 of a subject, according to prior art. PICC 100 is shown herein positioned in blood vessel 102, which is positioned in an upper arm 104 of the subject, is configured to deliver medical substance (not shown) through blood vessel 102 into a body organ, such as heart 108.

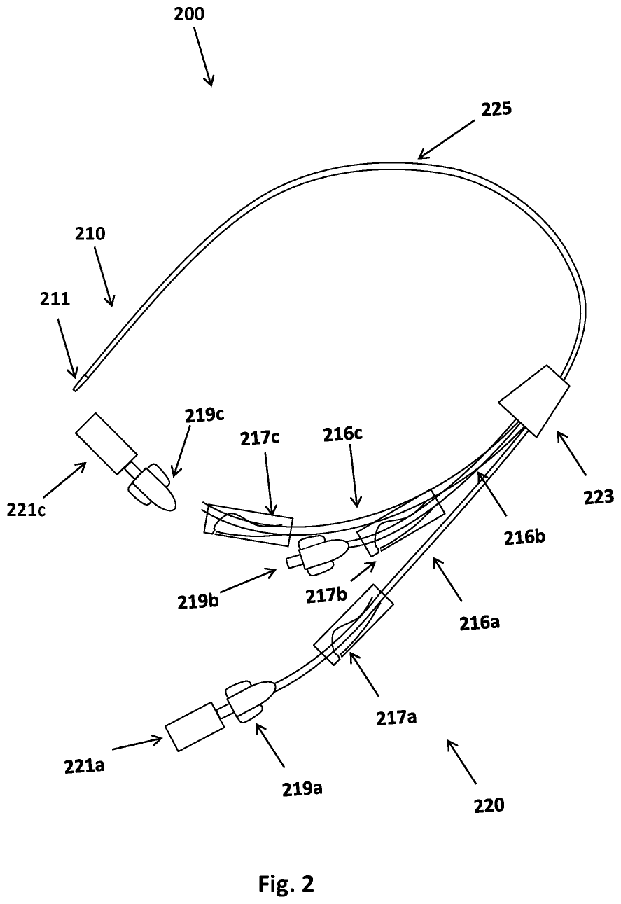

Reference is now made to FIG. 2, which schematically shows a standard PICC 200, according to prior art. Standard PICC 200 has a distal portion 210, configured to be inserted into a blood vessel. Optionally, distal portion 210 may include a soft tip 211, for minimizing trauma risk of the blood vessel. A proximal portion 220 of the PICC 200 may optionally include three individual lumens, lumen 216a, 216b and 216c. Each of lumens 216a, 216b and 216c is associated with an adapter 223 configured to connect a catheter line 225 with each of the lumens 216a, 216b and 216c. Each of lumens 216a, 216b and 216c is equipped with each of clamps 217a, 217b and 217c, respectively, configured to allow a temporarily scaling (e.g., for several minutes, hours or days) at the proximal end 220 without removing PICC 200 from the blood vessel. Further, each of lumens 216a, 216b and 216c may be equipped with each of connectors 219a, 219b and 219c. Connectors 219a, 219b and 219c may be needleless connectors, allowing a temporarily association with IV and/or syringes and/or external tubes or lumens, configured to deliver medical substances and/or other fluids (e.g., flushing fluid) into PICC 200. As shown in FIG. 2, connectors 219a and 219c may be associated with removable caps 221a and 221c for reducing infection risks. For example, a syringe (not shown) may be associated with lumen 216a via connector 219a at the proximal portion of the PICC 200. Then, upon opening clamp 217, a medical substance from the syringe may be administered into PICC 200 by flowing through adapter 223, catheter line 225 and exiting into the blood vessel via distal portion 210. Following the administration of medical substances, and especially in scenarios where administration of several types of medical substances is required, flushing of the catheter is performed for removing contaminations, bacteria, and the like. Flushing procedure is usually performed by inserting a flushing fluid, such as a saline solution, into one of the lumens (e.g., lumen 216c) at the proximal end 220 of PICC 200. Upon reaching the distal portion 210, the flushing fluid, including the contaminations, clotting factors, bacteria, and the like, is delivered into the blood vessel and/or body organ of the subject. Disposing the contaminations into the subject leads to an increased risk of infections and health problems.

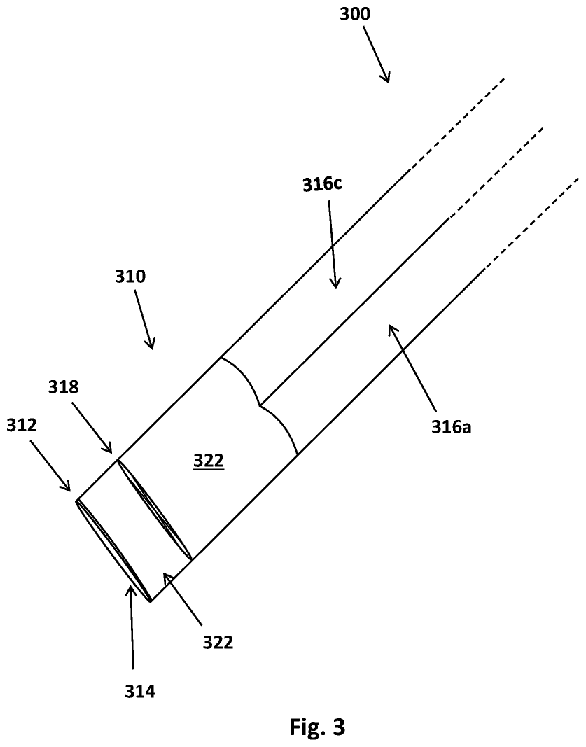

Reference is now made to FIG. 3, which shows a schematic illustration of a distal portion 310 of a PICC 300, according to some embodiments. PICC 300 is designed to facilitate circulation of flushing fluid such that the drained fluid disposal into a body organ is prevented. Hence, may limit, reduce, or eliminate bacteria, contaminations and/or precipitates which are administered from the flushing fluid into the subject. According to some embodiments of the current disclosure, distal portion 310 includes a distal end face 312, a distal opening 314, individual lumens 316a and 316c, and a valve 318. Lumens 316a and 316c extend along PICC 300 from a proximal portion thereof (not shown) to distal portion 310. As seen in FIG. 3, lumens 316a and 316c do not reach all the way to distal end face 312 forming a cavity 322 defined by an inner wall of PICC 300, the distal end of lumens 316a and 316c and valve 318. According to some embodiments, valve 318 is positioned inside cavity 322. According to some embodiments, flushing fluid may be inserted from a proximal end (not shown) of PICC 300 through lumen 316a, then, when valve 318 is in a closed configuration, the flushing fluid is circulated at distal portion 310 and drained from a proximal end (not shown) outside of PICC 300 through lumen 316c. According to some embodiments, flushing fluid may be inserted through the proximal end (not shown) of lumen 316c, circulated at distal portion 310, and then drained from a proximal end (not shown) outside of PICC 300 through lumen 316a. In other embodiments, flushing fluid may be drained out from each of lumens 316a and 316c and out of the body.

According to some embodiments, a cross-sectional size (e.g., for example, diameter) of lumen 316a may be different and/or equal to a cross-sectional size of the lumen 316c. According to some embodiments, the cross-sectional sizes of the lumens 316a/316c may correlate with a flow rate of fluid flowing therein. According to some embodiments, the cross-sectional sizes of the lumens 316a/316c may be different. According to some embodiments, the cross-sectional sizes of the lumens 316a/316c may vary. According to some embodiments, different configurations in which lumens 316a and 316c have different cross-sectional sizes may induce pressure differences between lumens 316a and 316c, thereby enabling a desirable flow rate in each of the lumens.

According to some embodiments, a wall thickness of each of the lumens 316a/316c may be different. According to some embodiments, the wall thickness of at least one of the lumens 316a/316c may vary. Advantageously, having different and/or varying wall thicknesses for the lumens 316a/316c may allow desirable pressure differences of fluid flow therein. According to some embodiments, having different and/or varying wall thicknesses for the lumens 316a/316c may increase the durability of the PICC.

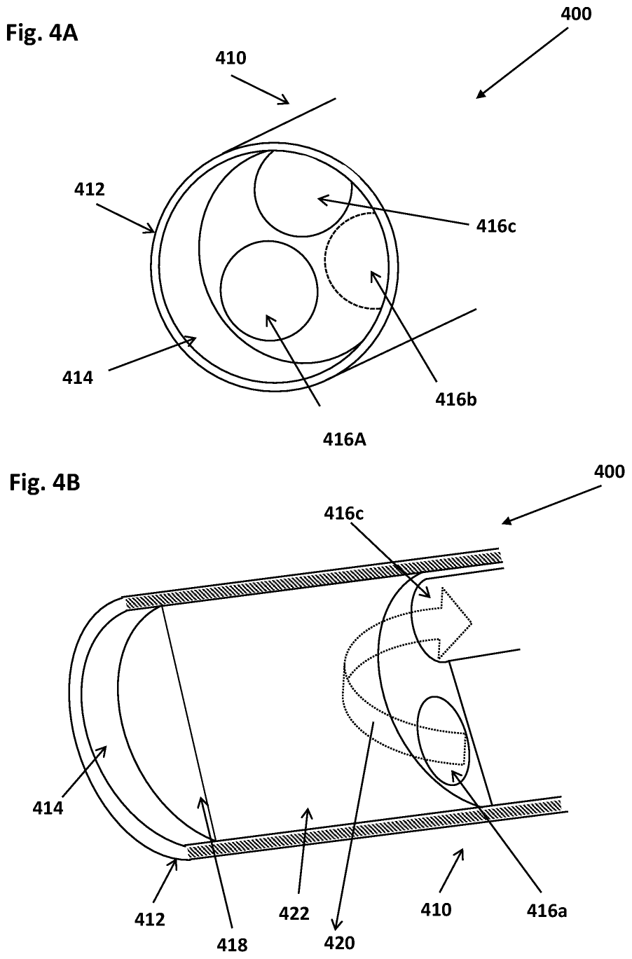

Reference is now made to FIG. 4A and FIG. 4B, which show a schematic illustration of a perspective view of a distal portion 410 of a PICC 400 and show a cross-section view of distal portion 410 and distal end face 412 of PICC 400, according to some embodiments. Distal portion 410 includes a distal opening 414 located at a distal end face 412 of PICC 400. PICC 400 includes an individual lumen 416a, an individual lumen 416c, and, optionally, may also include an individual lumen 416b. According to some embodiments, lumen 416b may include one or more individual lumens. Lumens 416a, 416b and 416c extend along PICC 400 from a proximal portion thereof (not shown) to distal portion 410. As also seen in FIG. 3, lumens 416a, 416b and 416c do not reach all the way to distal end face 412 forming a cavity 422 defined by an inner wall of PICC 400, the distal end of lumens 416a, 416b and 416c and a valve 418 (as depicted in FIG. 4B). According to some embodiments, lumen 416a may be utilized as a medical substance administration rout, and lumens 416a and 416c may be utilized as flushing lumens, while the lumen 416b may be utilized as the service lumen for distal valve (such as valve 418, as depicted in FIG. 4B) operating. In other words, lumen 416a may serve both as a medical substance administrator and as a flushing lumen. According to some embodiments, each of lumens 416a-c may, independently, be implemented as the medical substance administrator, and/or as the flushing lumen and/or as the service lumen. According to some embodiments where the service lumen is present (such as lumen 416b), it may be advantageous to utilize the service lumen for valve operating only.

Reference is now made to FIG. 4B, which shows a schematic illustration of a cross-sectional view of distal portion 410 of PICC 400 depicted in FIG. 4A, according to some embodiments. According to some embodiments, PICC 400 includes a plurality of individual lumens, an individual lumen 416a and an individual lumen 416c. FIG. 4B schematically depicts a flow direction 420 of a flushing fluid introduced, for example, into lumen 416a from the proximal end face of PICC 400 (not shown). As depicted by FIG. 4B, closed configuration of a valve 418 results in circulation of the flushing fluid, due to a flow of fluid from lumen 416a into lumen 416c. The fluid from lumen 416a flows from the proximal end of the PICC towards the valve 418 (and/or towards the distal end of the PICC). The valve 418, at a closed configuration thereof, constricts the flow of the fluid from lumen 416a from exiting the distal end of the PICC, thereby forcing the circulation of the fluid into lumen 416c, as depicted by arrow 420. Consequently, the closed configuration of valve 418 prevents entering of the flushing fluid into the blood vessel/body organ, and allows draining of the flushing fluid outside of the blood vessel and/or body organ. Hence, reducing the risk of infections and other medical complications.

According to some embodiments, the plurality of individual lumens may include one or more valves, such as duckbill valves (not shown). The one or more valves are configured to prevent entering of the circulated fluid into the distal end of one or more of the individual lumens and flow towards the proximal end therein. As a non-limiting example, a PICC 400 may include a plurality of lumens, such as four individual lumens. A first lumen 416a (i.e., flushing lumen) configured to deliver the flushing fluid, a second lumen 416c (i.e., flushing lumen) configured to receive the flushing fluid and direct it to the distal end and outside the subject, and a lumen 416b which includes two individual lumens, a first individual lumen (not shown) optionally configured to operate a valve 418, and a second individual lumen (not shown), which includes the one or more valves and, optionally, configured to deliver medical substance which should not be mixed with other substances and/or fluids. When valve 418 shifts from open to closed configuration, flushing fluid entering the first lumen 416a is circulated inside a cavity 422, i.e., the flushing fluid changes the flow direction from the distal portion 410 towards the proximal portion (not shown) of PICC. Hence, to avoid entering of the circulated fluid into the second individual lumen, the second individual lumen may include the one or more valves. According to some embodiments, the one or more valves may be positioned inside the second individual lumen. According to some embodiments, each of the plurality of lumens may include one or more valves.

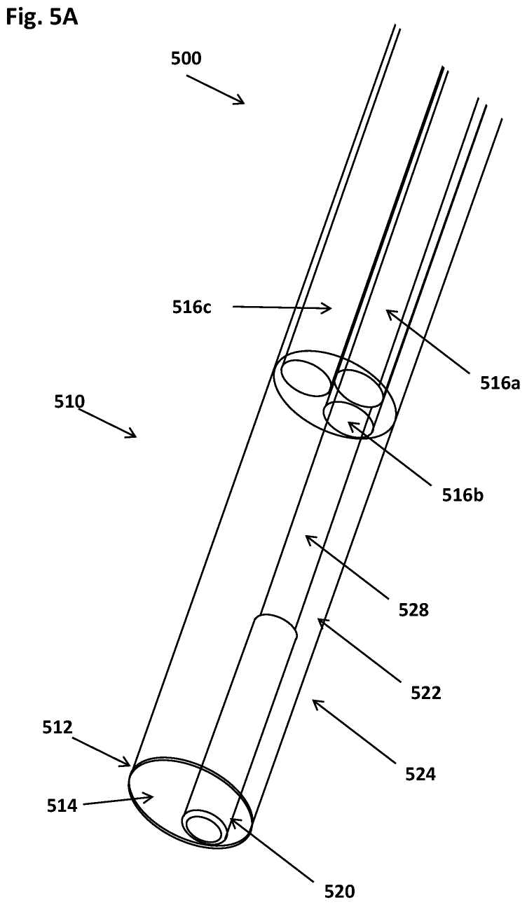

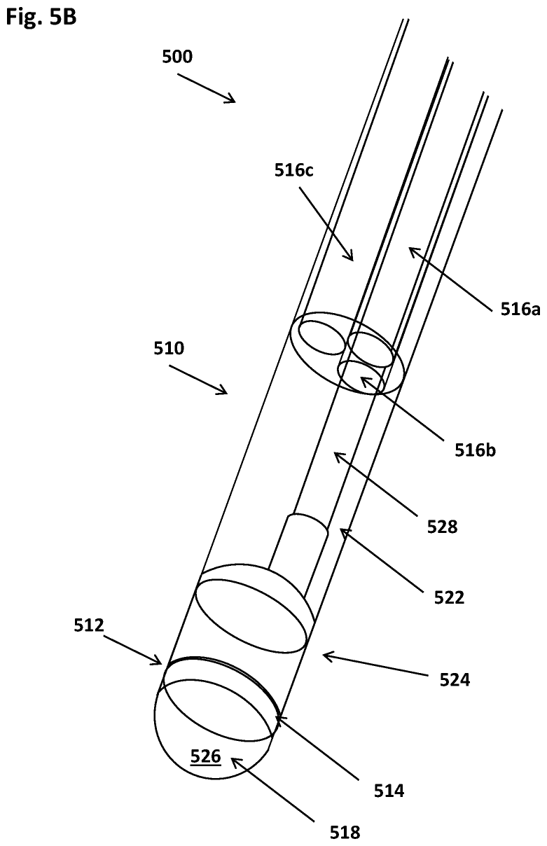

Reference is now made to FIGS. 5A and 5B, which show a schematic perspective view of a distal portion 510 of a PICC 500 in an open valve configuration 520 (depicted in FIG. 5A) and in a closed valve configuration 518 (depicted in FIG. 5B), according to some embodiments. Distal portion 510 includes a distal opening 514 located at a distal end face 512 of PICC 500. According to some embodiments, PICC 500 includes an individual lumen 516a, an individual lumen 516b and an individual lumen 516c, a cavity 522 formed between the distal end of the lumens 516b and 516c and the distal end face 512 of PICC 500, and a valve 524 positioned inside the cavity 522. According to some embodiments, lumen 516c may serve as a service lumen and may include a service conduit 528. In some embodiments, service conduit 528 may control valve 522 configuration shifting (e.g., from closed valve configurating 518 to open valve configuration 520, and vice versa). In some embodiments, service conduit 528 may assist during administration of PICC 500 into a blood vessel and/or body organ. For example, while catheters are typically made of soft materials which are not visible at X-ray imaging, service conduit 528 may be made of materials that assist viability and stability during X-ray, thus facilitating easier and proper positioning of PICC 500 inside a blood vessel and/or body organ. As depicted in FIG. 5A, the open valve configuration 520 allows administrating of medical substance into the blood vessel and/or body organ of the subject.

As depicted in FIG. 5B, the valve 524 may include a balloon 526. According to some embodiments, at a closed configuration 518, the valve 524 (or the balloon 526) may be at least partially inflated. According to some embodiments, at a closed configuration 518, the valve 524 may be inflated such that the balloon 526 obstructs the distal end of the PICC. According to some embodiments, the PICC may include a service conduit 528 configured to deliver fluid (e.g., for example, a gas) to and/or from the balloon 526. According to some embodiments, the service conduit 528 may be in fluid communication with the balloon 526. According to some embodiments, the service conduit 528 may be positioned within the lumen 516c. According to some embodiments, the service conduit 528 may be positioned on an outer wall and/or abutting the PICC. Advantageously, an inflated balloon valve may prevent the flushing fluid from exiting the distal opening 514 and entering the blood vessel and/or body organ. Advantageously, when the PICC is idle and the balloon 526 of valve 524 is inflated, it may prevent infections dwelling back from the body to one or more lumens.

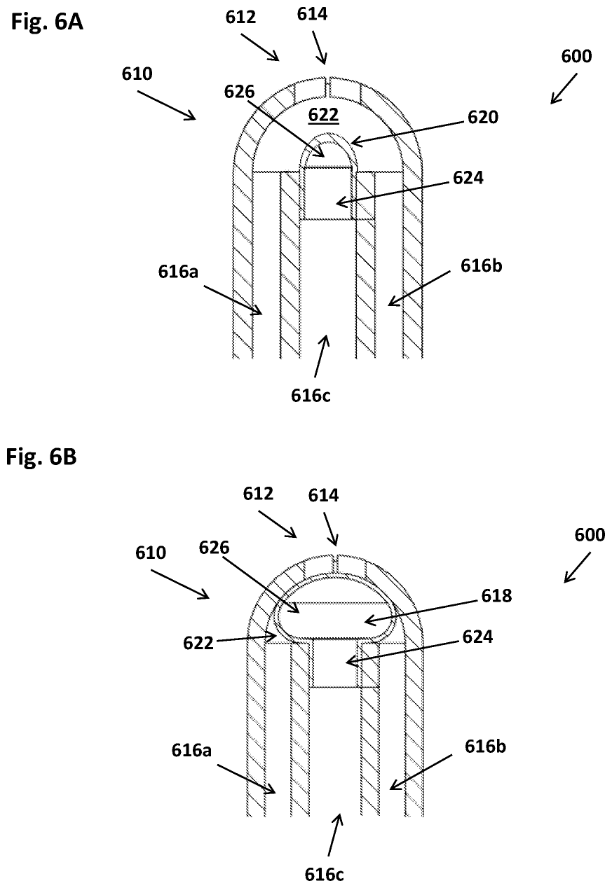

Reference is now made to FIGS. 6A and 6B, which show a schematic cross-sectional side view of a distal portion 610 of a PICC 600 in an open valve configuration 620 (depicted in FIG. 6A) and in a closed valve configuration 618 (depicted in FIG. 6B), according to some embodiments.

According to some embodiments, the following components/features depicted in FIGS. 6A and 6B 600, 610, 612, 614, 616a-c, 622 and 624 correspond to and may have identical or similar structure, configuration and/or characteristics as the previously described herein components 500, 510, 512, 514, 516a-c, 522 and 524 in FIG. 5.

According to some embodiments and as depicted in FIGS. 6A and 6B, a distal portion 610 of the PICC 600 may include, among others, a circular or circular-like structure. Alternatively, the distal portion 610 of the PICC 600 may include an angle-shaped structure, such as but not limited to, conus-like, rectangular, polynomic structure, and the like or any combination thereof. According to some embodiments, PICC 600 includes an individual lumen 616a, an individual lumen 616b and an individual lumen 616c. According to some embodiments, PICC 600 may include 2 individual lumens (e.g., individual lumens 616a and 616b). According to some embodiments, PICC 600 may include 3 or more individual lumens. Each possibility is a separate embodiment.

According to some embodiments, PICC 600 includes a cavity 622 formed between a distal end of the individual lumens 616ba, 616b and 616c and a distal end face 512 of PICC 600, and a valve 624 positioned inside the cavity 622. According to some embodiments, the valve 624 includes an inflatable element, such as a balloon 626 configured to inflate into dimensions sufficient for scaling a distal opening 614, thus preventing fluid flow into and/or from a blood vessel and/or body organ. According to some embodiments, the PICC 600 may include an inflation conduit (e.g., in the individual lumen 616c) in fluid flow communication with the inflatable element. According to some embodiments, inflation and deflation of the inflatable element may be performed via the inflation conduit. According to some embodiments, the balloon 626 may be inflated with fluid, such as but not limited to, saline solution. According to some embodiments, the inflation of the balloon 626 may be performed at a proximal portion (not shown) of the PICC 600.

According to some embodiments and as depicted in FIGS. 6A and 6B, the balloon 626 is partly positioned within the cavity 622 and partially inside the individual lumen 616c. According to some embodiments, the balloon 626 may be positioned outside the individual lumen 616c. According to some embodiments, the balloon 626 may be positioned outside the individual lumen 616c. As a non-limiting example, the balloon 626 may be positioned on an outer portion of the individual lumen 616c. As yet another non-limiting example, the balloon 626 may be essentially positioned within the cavity 622.

According to some embodiments and as depicted in FIG. 6B, the closed valve configuration 618 of the balloon 626 includes at least a partial inflation thereof, essentially filling the cavity 622, thereby preventing fluid from there though. Additionally or alternatively, in some embodiments, the closed valve configuration 618 may include filling the cavity 626 as well as filling a distal portion of each of the individual lumens 616a and 616b.

According to some embodiments, the closed valve configuration 618 includes at least partial inflation of the balloon 626 extending outside the distal end face 612. According to some embodiments, the closed valve configuration 618 the balloon 626 is inflated by occupying only the inter components of the PICC 600 (e.g., by filling the cavity 622).

According to some embodiments, the individual lumen 616c is a service lumen configured to control and operate the configuration shifting (i.e. open/closed) of the valve 624. According to some embodiments, the individual lumen 616c is a service lumen configured to control the inflated/deflated configuration of the balloon 626. According to some embodiments, PICC 600 may include or be associated with a service conduit positioned inside the individual lumen 616c. According to some embodiments, the service conduit may be in fluid communication with the valve 624. According to some embodiments, the service conduit may control the configuration shifting of the valve 624 (e.g., from closed valve configuration 618 to open valve configuration 620, and vice versa). In some embodiments, the service conduit may assist in administration of PICC 600 into a blood vessel and/or body organ.

According to some embodiments, the valve shifting mechanism may be controlled and/or performed manually, e.g., by a caregiver and/or by the subject by mechanically operating the valve 624 at a proximal end of the PICC 600. Alternatively or additionally the valve shifting mechanism may be performed automatically, for example, by sensors control.

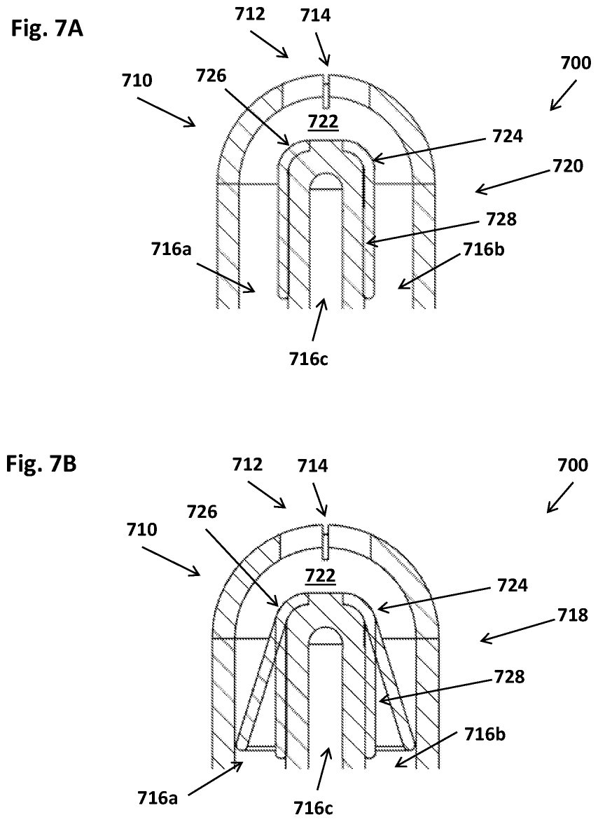

Reference is now made to FIGS. 7A and 7B, which show a schematic cross-sectional side view of a distal portion 710 of a PICC 700 in an open valve configuration 720 (depicted in FIG. 7A) and in a closed valve configuration 718 (depicted in FIG. 7B), according to some embodiments.

According to some embodiments, the following components/features depicted in FIGS. 7A and 7B 700, 710, 712, 714 and 716a-c, correspond to and may have identical or similar structure, configuration and/or characteristics as the previously described herein components 600, 610, 612, 614 and 616a-c in FIGS. 6A and 6B.

According to some embodiments, a cavity 722 is formed between a distal end of an individual lumens 716a and 716c and a distal end face 712 of PICC 600, and a valve 724 positioned inside the cavity 722.

According to some embodiments, the valve 724 includes a scaling flap and/or a shutter configured to controllably seal the distal end face opening. According to some embodiments, the sealing flap and/or the shutter may be in a form of a skirt valve 726.

According to some embodiments, the valve 724 includes a skirt valve 726 configured to controllably seal a distal opening 714. According to some embodiments, the skirt valve 726 may be fused or otherwise integrally formed on a distal end of the outer surface of the individual lumen 716c. According to some embodiments, the skirt valve 726 may be detachably associated with the individual lumen 716c. According to some embodiments, the skirt valve 726 includes a rim 728, such that in the open valve configuration 720 the rim 728 is essentially vertically positioned, allowing fluid flow therethrough, and in the closed valve configuration 718 the rim 728 is in a downwardly sloping position, thereby preventing passing of fluids therethrough. Alternatively or additionally, in some embodiments, the closed valve configuration 718 may include an upwardly sloping position. According to some embodiments and as depicted in FIG. 7B, scaling of the PICC 900 by the skirt valve 826 includes an internal sealing of each of the individual lumens 716a and 716b. According to some embodiments, the closed configuration 718 of the skirt valve 726 may include scaling the cavity 722.

According to some embodiments, without wishing to be bound to any specific mechanism, the skirt valve 726 may be operated via a service conduit (not shown) positioned within the individual lumen 716c. As a non-limiting example, the service conduit may be associated, among others, with a wire such that upon pushing the wire distally the rim 728 is shifted into the downwardly sloping position (i.e. the closed configuration 718), sealing the distal end face 712 of the PICC 700, and upon pulling the wire proximately the rim 728 is shifted into an essentially vertical position, (i.e. the open configuration 720), thereby allowing fluid flow through a distal opening of the distal end face of the PICC.

According to some embodiments, the configuration of the skirt valve 726 may be mechanically or manually (e.g., by a subject and/or a caregiver) operated from a proximal end of the PICC 700, and/or automatically operated, for example, by sensors control. As a non-limiting example, the skirt valve 726 configuration may be controlled by operating a service conduit (not shown) positioned within the individual lumen 716c.

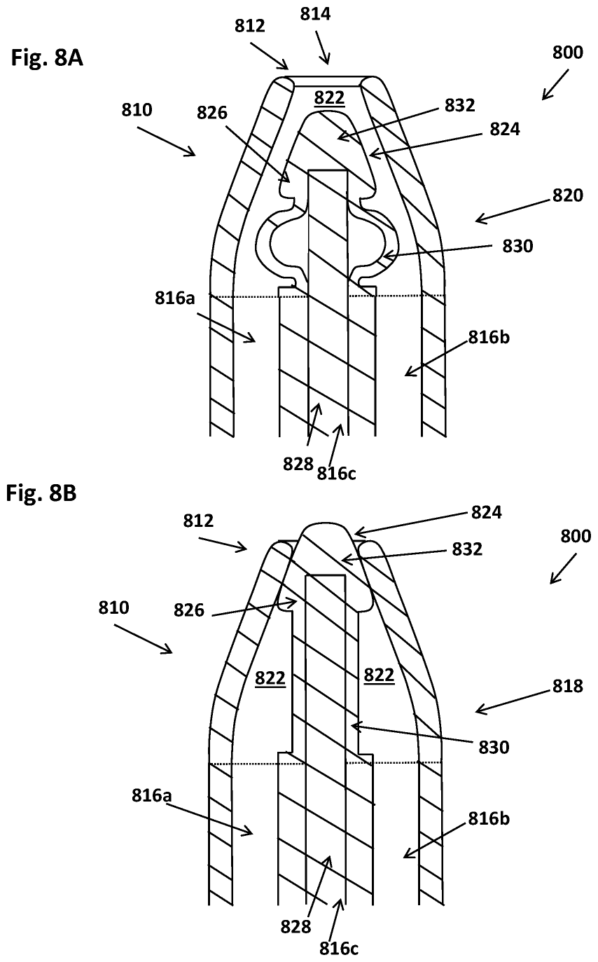

Reference is now made to FIGS. 8A and 8B, which show a schematic cross-sectional side view of a distal portion 810 of a PICC 800 in an open valve configuration 820 (depicted in FIG. 8A) and in a closed valve configuration 818 (depicted in FIG. 8B), according to some embodiments.

According to some embodiments and as depicted in FIGS. 8A and 8B, a distal portion 810 of the PICC 800 includes an angle-shaped structure, such as but not limited to, conus-like structure. Alternatively, in some embodiments, the distal portion 810 may include a circular or circular-like structure. According to some embodiments, PICC 800 includes an individual lumen 816a, an individual lumen 816b and an individual lumen 816c. According to some embodiments, the individual lumen 816c is a service lumen. According to some embodiments, PICC 800 may include two individual lumens. According to some embodiments, PICC 800 may include three or more individual lumens. Each possibly is a separate embodiment.

According to some embodiments, a cavity 822 is formed between a distal end of the individual lumens 816a, 816b and 816c and a distal end face 812 of PICC 800, and a valve 824 is positioned inside the cavity 822.

According to some embodiments, the valve 824 includes a guidewire valve 826 configured to controllably seal a distal opening 814. According to some embodiments, the guidewire valve 826 includes a cap 832, an elastic valve member 830, and is detachably or permanently associated with a guidewire 828.

According to some embodiments, the guidewire valve 826 is a valve operated by the guidewire 828 such that upon pushing the guidewire 828 distally the guidewire valve 826 is in the closed configuration 820, thereby sealing the distal end face 812 of the PICC 800, and upon pulling the guidewire 828 proximately the guidewire valve 826 is in the open configuration 818, thereby allowing fluid flow through the distal opening 814 of the distal end face 812.

According to some embodiments, the guidewire valve 826 may be fused or otherwise integrally formed on the outer surface of a distal end of the individual lumen 816c. As a non-limiting example, at least a portion of the elastic valve member 830 may be associated with the distal end of the individual lumen 816c. According to some embodiments, the guidewire valve 826 may be detachably associated with the individual lumen 816c.

According to some embodiments, the guidewire valve 826 may be inserted into the PICC 800 only when sealing thereof is required. Alternatively, in some embodiments, the guidewire valve 826 may be positioned within the PICC both at open and closed configurations 818 and 820, respectively, thereof.

According to some embodiments, the guidewire valve 826 includes or made of flexible, elastic and/or soft materials, such as but not limited to, polymers. According to some embodiments, the cap 832 and the elastic member 830 may include or made of different materials than the guidewire 828. According to some embodiments, the guidewire 828 may include or be made of materials having lower flexibility relative to the cap 832 and/or the elastic member 830. According to some embodiments, the guidewire 828 may include or be made of metals, such as but not limited to, aluminum.

According to some embodiments, the cap 832 is configured to seal the distal end face 812 of the PICC 800, thus preventing fluid flow therethrough According to some embodiments, the structure of the cap 832 is geometrically complementary with the distal opening 814 and/or with at least a portion of the distal end face 812. According to some embodiments, the cap 832 includes, among others, an angular structure (e.g., pyramid-like, conus-like, and the like) to facilitate sealing the distal end face 812 of the PICC 800.

According to some embodiments, the elastic valve member 830 is positioned on the outer portion of a lumen, e.g., the individual lumen 816c. Alternatively or additionally, in some embodiments, at least a portion of the elastic valve member 830 may be positioned inside the individual lumen 816c. According to some embodiments, the guidewire valve 826 is associated with a guidewire 828 positioned inside the individual lumen 816c, allowing shifting the configuration of the guidewire valve 826.

According to some embodiments and as depicted in FIG. 8A, in the open valve configuration, the elastic valve member 830 is at least partially folded, allowing passage of fluids through the distal opening 814 of the distal end face 812 of the PICC 800. According to some embodiments, in the open valve configuration 818 the cap 832 is at least partially positioned inside the cavity 822. According to some embodiments and as depicted in FIG. 8B, in the closed valve configuration, the clastic valve member 830 is pushed distally (e.g., by extending, unfolding and the like of the elastic member 830) such that the cap 832 seals the distal end face 812 of the PICC 800.

According to some embodiments, the configuration of the guidewire valve 826 may be mechanically operated from a proximal end of the PICC 800, and/or automatically operated, for example, by sensors control. According to some embodiments, implementing the guidewire valve 826 advantageously allows technically simple operation of the PICC 800 and reduces clogging risks.

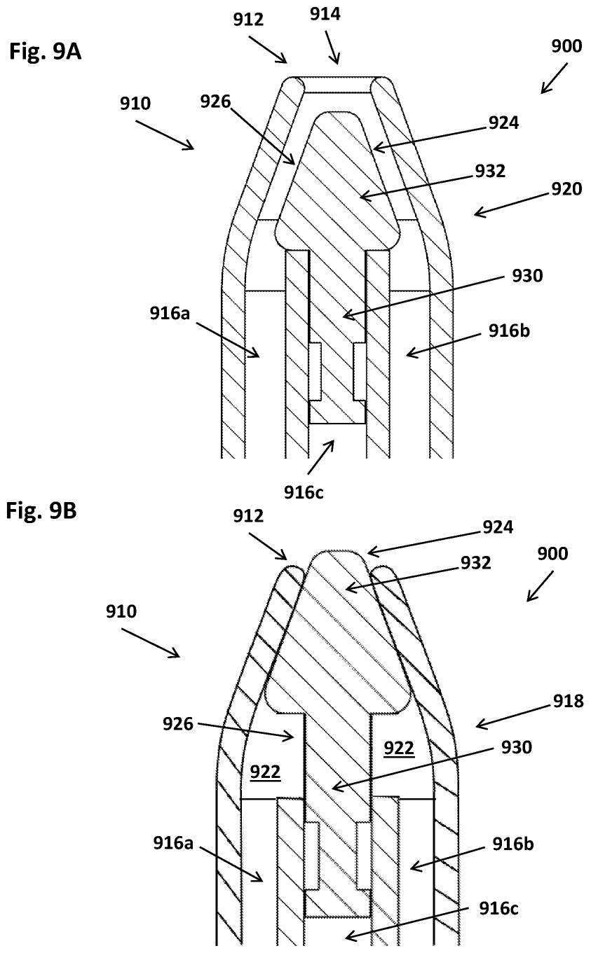

Reference is now made to FIGS. 9A and 9B, which show a schematic cross-sectional side view of a distal portion 910 of a PICC 900 in an open valve configuration 920 (depicted in FIG. 9A) and in a closed valve configuration 918 (depicted in FIG. 9B), according to some embodiments.

According to some embodiments, the following components/features depicted in FIGS. 9A and 9B 900, 910, 912, 914 and 916a-c, correspond to and may have identical or similar structure, configuration and/or characteristics as the previously described herein components 800, 810, 812, 814 and 816a-c in FIGS. 8A and 8B.

According to some embodiments, a cavity 922 is formed between a distal end of individual lumens 916a, 916b and 916c and a distal end face 912 of PICC 900, and a valve 924 is positioned inside the cavity 922.

According to some embodiments, the valve 924 is includes a piston 926 configured to controllably seal a distal opening 914. According to some embodiments and as depicted in FIGS. 9A and 9B, the piston 926 is partially positioned within the individual lumen 916c. According to some embodiments, the piston 926 includes or made of rigid or semi-rigid materials, such as but not limited to, polymers, metals, and the like.

According to some embodiments, the piston 926 may be operated by a wire or any other suitable pushing means (not shown) such that upon pushing the wire distally the piston 926 is shifted to the closed configuration 920, thereby sealing a distal end face 912 of the PICC 900, and upon pulling the wire proximately the piston 926 is shifted to the open configuration 918, thereby allowing fluid flow through the distal opening 914 of the distal end face 912.

According to some embodiments, the piston 926 includes a body 930 and a cap 932. According to some embodiments, at least a portion of the body 930 of the piston 926 is configured to fit inside a lumen e.g., a service lumen, e.g., the individual human 916c. According to some embodiments, the piston 926 is advantageously positioned within the individual lumen 916c, minimizing the risks of infections, clogging, bacteria accumulation, and the like. According to some embodiments, the cap 932 includes an angular structure, such as but not limited to, a pyramid, conus, and the like, configured to seal the distal end face 912 of the PICC 900. According to some embodiments, the cap 932, in the closed configuration 918, is at least partially extended from the distal end face 912. According to some embodiments, the piston 926 operation is advantageously independent of flushing pressure within the individual lumen 916c.

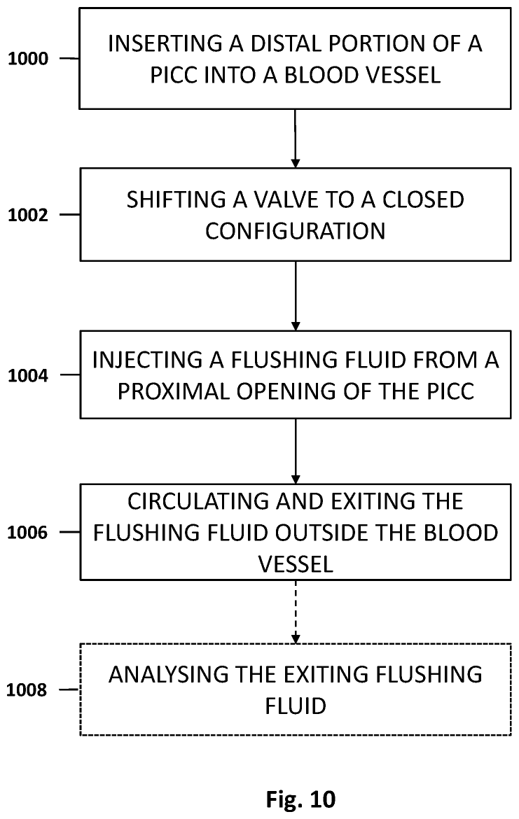

According to some embodiments, the piston 926 is at least partially positioned within the individual lumen 916c at the open and/or the closed configurations 920 and 918, respectively, thereof. According to some embodiments, the piston 926 advantageously provides operational certainty (e.g., by minimizing inadvertent pushing/pulling or otherwise displacing thereof) of each of the open and the closed configurations 920 and 918, respectively. Reference is now made to FIG. 10, which shows a flow chart of a method of flushing of a PICC, according to some embodiments, such as but not limited to, PICCs 300, 400 and 500.

At step 1000, the method includes inserting a distal portion of a PICC into a blood vessel. According to some embodiments, a caregiver inserts a distal portion (such as distal portion 310, 410, 510, 610, 710, 810 or 910 of the PICC 300, 400, 500, 600, 700, 800 or 900) into a blood vessel and/or organ of a subject. Optionally, the inserting may be performed by using a guidewire, e.g., nitinol guidewire, and/or by imaging devices, such as X-ray, to validate the proper locating of a PICC inside the subject.

At step 1002, the method includes shifting a valve to a closed configuration. According to some embodiments, the valve located at the distal portion of the PICC is shifted to a closed configuration. According to some embodiments, valve may include, but is not limited to, an inflatable balloon, which, when inflated, seals the distal portion of the PICC. Valve shifting mechanism may be controlled manually, e.g., by a caregiver and/or the subject by mechanically operating the valve at a proximal end, and/or automatically, for example, by sensors control.

At step 1004, the method includes injecting a flushing fluid from a proximal opening of the PICC. According to some embodiments, the flushing fluid, such as saline, may be inserted into the catheter from the proximal opening.

At step 1006, the method includes circulating and exiting the flushing fluid outside the blood vessel. According to some embodiments, the flushing fluid continues to flow until reaching the distal portion of the PICC. Upon reaching the closed valve configuration, flow direction of the flushing fluid changes-circulates, and the flushing fluid enters a different lumen, allowing the flushing fluid flow from the distal portion to the proximal portion of the catheter and outside the body. In some embodiments, the flushing fluid may be circulated and enter the same lumen.

At step 1008, the method includes analysing the exiting flushing fluid. According to some embodiments, upon exiting of the flushing fluid, the flushing fluid may be collected, and optionally, further analysed for presence of any chemical and/or biological substances, such as, for example, contaminations, precipitates, bacteria, and the like. According to some embodiments, presence of chemical and/or biological substances may be indicative of complications and/or infections. According to some embodiments, analysis of the exiting flushing fluid allows identification of blood clots and/or bacteria. According to some embodiments, identification of bacteria presence may assist the medical staff in providing proper treatment to the subject, thereby increasing health status and wellness of the subject.

According to some embodiments, the method may include analyzing one or more parameters associated with a pressure of the flushing fluid exiting and/or entering the catheter for assessing the performance quality of the catheter and/or the flushing fluid, as well as may assist with measuring the quantities of medical substance administered. In some embodiments, exceeding the maximum allowed pressure inside the PICC leads to formation of various defects, such as kinks, which may decrease the integrity of the PICC. According to some embodiments, pressure changes inside the PICC may indicate presence of precipitates and/or accumulation of residue. Accumulation of undesired materials and obstructions inside the PICC may eventually lead to blockage of the PICC. According to some embodiments, the method may include monitoring pressure of the medical substance and/or flushing fluid using one or more sensors. One or more sensors may be located at various positions across the length of the PICC, for example, at the distal end and/or the proximal end. According to some embodiments, one or more sensors may be associated with one or more of the lumens. According to some embodiments, the one or more sensors may be configured to monitor the pressure of the medical substance and/or flushing fluid within one or more of the lumens. In some embodiments, it may be preferable to place the one or more sensors such that a smooth inner and/or outer surface of the lumens and/or the PICC is maintained, thereby reducing a risk of infections.

According to some embodiments, the method may include monitoring flow capacity of the medical substance entering the blood vessel using one or more sensors. According to some embodiments, changes in flow capacity may indicate malfunction of the PICC, and, optionally, may require a replacement of the PICC.

According to some embodiments, the method may include monitoring flow capacity of the flushing fluid entering and/or exiting the catheter using one or more sensors. According to some embodiments, changes in the flow capacity of the flushing fluid may indicate valve malfunction and/or replacement. According to some embodiments, valve replacement may be performed by a service conduit.

According to some embodiments, the method may include regulating, utilizing a controller, pressure and/or flow capacity of the flushing fluid and/or the medical substance. Consequently, facilitating the maintaining of pressure and/or flow capacity in the optimal range, thus increasing integrity of the PICC.

In the description and claims of the application, the words “include” and “have”, and forms thereof, are not limited to members in a list with which the words may be associated.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although stages of methods according to some embodiments may be described in a specific sequence, methods of the disclosure may include some or all of the described stages carried out in a different order. A method of the disclosure may include a few of the stages described or all of the stages described. No particular stage in a disclosed method is to be considered an essential stage of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

1. A peripherally insertable central catheter (PICC) comprising
a distal portion, configured to be inserted into a blood vessel, the distal portion comprises a distal end face opening;
a proximal portion comprising a proximal opening;
at least one flushing lumen extended therebetween; and
a valve positioned within the at least one flushing lumen, the valve comprises two configurations, an open configuration, allowing fluid flow through the distal end face opening, and a closed configuration, blocking fluid flow through the distal end face opening, wherein at a closed configuration of the valve, flushing fluid that is inserted to the catheter from the proximal opening thereof, circulates back through the proximal portion and out of the subject's blood vessel, thereby exiting of flushing fluid through the distal end face opening is prevented.

2. The catheter of claim 1, wherein the at least one flushing lumen is further configured to deliver a medical substance to the subject at an open configuration of the valve.

3. The catheter of claim 1, wherein the at least one flushing lumen is divided into two individual lumens, a first lumen configured to deliver the flushing fluid and a second lumen configured to collect the flushing fluid and direct it to the proximal portion and out of the subject's blood vessel.

4. The catheter of claim 1, wherein the valve comprises an inflatable element configured to inflate into dimensions sufficient for sealing the distal end face opening.

5. The catheter of claim 4, further comprising an inflation conduit in fluid flow communication with the inflatable element.

6. The catheter of claim 1, wherein the valve comprises a sealing flap and/or a shutter configured to controllably seal the distal end face opening.

7. The device of claim 1, wherein the valve comprises a guidewire valve, a piston, a duckbill valve or a skirt valve configured to controllably seal the distal end face opening.

8. (canceled)

9. (canceled)

10. (canceled)

11. The catheter of claim 1, further comprising an additional individual lumen for delivering a medical substance to the subject at an open configuration of the valve.

12. The catheter of claim 1, wherein the flushing fluid circulated back through the proximal portion exits the catheter through the proximal opening and/or through a proximal drain port.

13. (canceled)

14. The catheter of claim 1, further comprising a pressure adjustment mechanism configured to regulate a flow rate of the flushing fluid to facilitate circulation of the flushing fluid through the at least one flushing lumen, back through the proximal portion and out of the catheter.

15. The catheter of claim 1, comprising a flushing lumen, a lumen for operating the valve, a lumen for delivering medical substance, and a lumen comprising a duckbill valve for facilitating the separation of the medical substances,

16. The catheter of claim 1, further comprising one or more sensors for monitoring and/or regulating: pressure of the flushing fluid to facilitate pressure regulation; flow capacity of the flushing fluid entering and/or exiting the catheter; and/or flow capacity of the medical substance entering the blood vessel.

17. (canceled)

18. (canceled)

19. A method for flushing a peripherally insertable central catheter (PICC), the method comprising:
providing a PICC comprising
a distal portion comprising a distal end face opening;
a proximal portion comprising a proximal opening;
at least one flushing lumen extended therebetween; and
a valve positioned within the at least one flushing lumen;
inserting the distal portion of the PICC into a blood vessel of a subject;
shifting the valve to a closed configuration for blocking fluid flow through the distal end face opening; and
injecting flushing fluid to the catheter from the proximal opening thereof, thereby the flushing fluid circulates back through the proximal portion and out of the catheter such that exiting of flushing fluid through the distal end face opening and into the subject's blood vessel is prevented.

20. The method of claim 19, further comprising analyzing the flushing fluid for chemical and/or biological substances indicative of complications and/or infections.

21. The method of claim 19, wherein the analyzing the flushing fluid comprises identification of blood clots and/or bacteria.

22. The method of claim 19, further comprising analyzing one or more parameters associated with a pressure of the flushing fluid exiting and/or entering the catheter for assessing the performance quality of the catheter and/or the flushing fluid.

23. The method of claim 19, further comprising monitoring and/or regulating pressure of the flushing fluid using one or more sensors.

24. The method of claim 19, further comprising monitoring and/or regulating flow capacity of the flushing fluid entering and/or exiting the catheter using one or more sensors.

25. The method of claim 19, further comprising monitoring and/or regulating flow capacity of the medical substance entering the blood vessel using one or more sensors.

26. The method of claim 19, further comprising regulating, utilizing a controller, pressure and/or flow capacity of the flushing fluid and/or the medical substance.
